(19) United States
(12) Patent Application Publication
(10) Pub. No.: US 2019/0366038 A1
(43) Pub. Date: Dec. 5, 2019

(54) URINARY CATHETER

(71) Applicant: Rainbow Medical Engineering Limited, Royston, Hertfordshire (GB)

(72) Inventors: Clive Denman, Lymington (GB); Luigi Gerard Martini, London (GB); Alan Lynford Profit, Old Harlow (GB); David Seaward, Warwick (GB)

(73) Assignee: Rainbow Medical Engineering Limited, Royston, Hertfordshire (GB)

(21) Appl. No.: 16/467,734

(22) PCT Filed: Dec. 8, 2017
(86) PCT No.: PCT/GB2017/053694

§ 371 Date: Jun. 7, 2019

(30) Foreign Application Priority Data
Dec. 8, 2016 (GB) 1620922.3
Nov. 27, 2017 (GB) 1719638.7

(51) Int. Cl. A61M 25/00; A61M 27/00

(52) U.S. Cl. CPC A61M 25/0017; A61M 2025/0078; A61M 25/0075; A61M 27/008

(57) ABSTRACT

There is described a urological device comprising one or more valves within a valve housing, external to the body when in use, with means for connection to an in-dwelling catheter and a means for connection to a urine collection device for the reduction of Catheter Associated Urinary Tract Infections (CAUTI's), the valve characterised by having a higher opening pressure than closing pressure, and the urological device having one or more valves movable from a closed position to an open position in response to applied urological/intra-abdominal pressure; and wherein the device is provided with a vent to prevent siphoning action from the urine collection device; and wherein the valve has a region of coaption and the number of valves is varied to provide opening pressures matched to one or more intra-abdominal pressures in one or more patient groups.

### FIELD OF THE INVENTION

The present invention relates to a novel urological device, such as a urinary catheter and a urinary catheter assembly; and methods related thereto.

### BACKGROUND

Typically, in-dwelling (“Foley”) urinary catheters are used as a matter of routine during many surgical procedures and by patients suffering from urinary incontinence or by disabled individuals like paraplegics or tetraplegics, who may have no control over urination. Catheterisation may be the only available way of managing urination. Urinary catheters exist in combination with bags for collecting the urine which can be emptied into a toilet. Alternatively they can be used with a manually operated valve to empty the bladder directly into a toilet, albeit this requires dexterity. The use of catheters and leg bags can be embarrassing, and can diminish patient dignity.

Indwelling urinary catheters are used both in hospital and at home. In hospitals the patients may be post-operative or post-trauma for reasons not related to the bladder and unable to leave their bed or even be unconscious. They may also be suffering from a wide range of bladder conditions. In these patients the usage is relatively short-term i.e. days or weeks. Home usage is more related to ageing e.g. incontinence or those with longer term conditions that affect mobility or the bladder e.g. paraplegia, multiple sclerosis, etc. Usage may extend for a period of years.

It is well known to use urinary drainage systems, e.g. urinary catheters, to drain urine from the bladder in various hospital and medical settings. Urinary drainage systems provide a pathway for urine to flow from the body of a patient into a urine receptacle, e.g. a urine collection bag. Such urinary drainage systems generally use an indwelling urinary catheter which is connected to the urine receptacle (e.g. “leg bag”). This system therefore provides a flow path for the urine from the patient's bladder to the urine receptacle.

In most urinary drainage systems a patient's bladder is continuously emptied. However, when a catheter drains continuously, a patient's bladder is unable to fill. This can cause the patient's bladder to lose muscle tone, which can itself lead to a loss of voluntary voiding of the bladder, causing the patient to experience incontinence.

Indwelling catheters include an inflatable balloon at their tip to prevent accidental removal. The eyelet through which the urine flows is above the balloon. This creates a stagnant sump of urine in which bacteria can readily flourish, leading to an infection.

Catheterisation itself can introduce microorganisms into the urinary tract and/or the bladder of a patient. Furthermore, when a healthy patient's bladder is regularly emptied the patient's bladder and urethra are flushed, such that any build-up of biofilm is flushed away. The build-up of a urethral biofilm is a haven for micro-organisms that can cause infections, such as urinary tract infections.

A loss of voluntary voiding of the bladder can lead directly to urinary tract infections. Natural flushing of the urethra and bladder is eliminated which would otherwise clear the build-up of biofilm. Hence micro-organisms can build up, leading to infections. When catheters drain continuously, the bladder is unable to fill and void, and this promotes infection.

It is known to provide catheters with manually operated valves to void the bladder, but these require a wearer or healthcare worker to operate these, and these do not void when there is a natural contraction of the detrusor muscle, and/or increased in abdominal pressure via squeezing. Currently available valves also require significant dexterity often not available in elderly patients.

Between 15% and 25% of patients admitted to hospital undergo short-term catheterisation, that is, a catheter is in place for less than seven days. However, between 10% and 30% of those patients develop a Catheter Associated Urinary Tract Infections (CAUTI). In adult critical care units 45%-85% of patients have an indwelling urethral catheter. The incidence of urinary tract infections for patients that undergo long-term catheterisation, i.e. for eight days or more, is significantly higher and can be as high as 50% of patients.

Bacteria colonizing the drainage bags of catheterized patients have been reported to be a source for outbreaks of resistant organisms in acute care. In nursing homes setting, the urine of residents with chronic indwelling catheters is the most common site of isolation of resistant gram negative organisms.

Therefore, there is an urgent need to alleviate this problem.

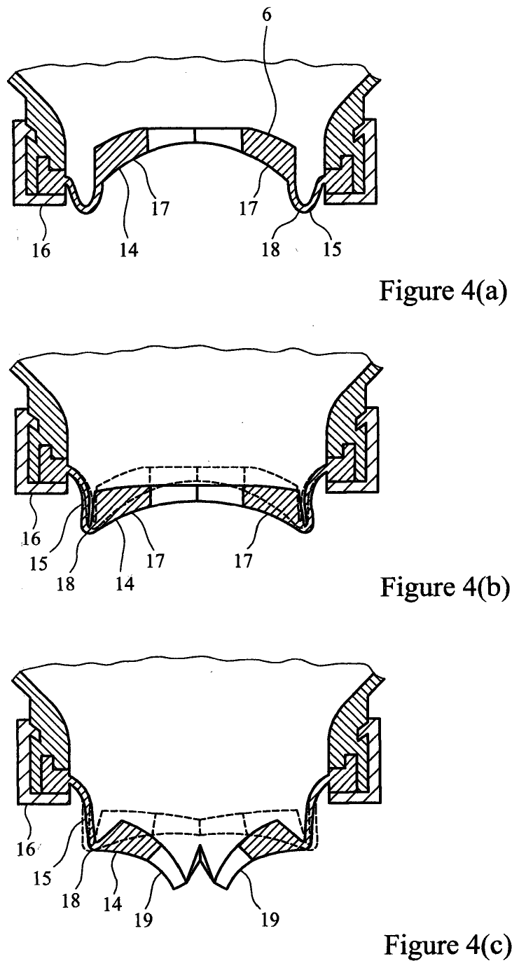

International Patent application No. WO 2011/073969 (Vysera Biomedical) attempts to address this by describing a urological device comprising a urological valve, wherein the valve comprising a plurality of valve leaflets. The valve is implantable within the body, and is openable in response to applied urological pressure. Whilst the valve described in WO 2011/073969 provides an advance on previous catheter arrangements, it provides a “one size fits all” implanted valve. Such a “one size fits all” valve is disadvantageous, since the valves are not tailored to individual needs or to the needs of specified patient groups. The urological pressure can vary depending upon the gender, age, condition, etc. of the patient (see FIG. 5).

The present invention seeks to provide a solution to this by providing a urological device with a valve that has an opening pressure that is matched to different intra-abdominal pressures in different patient groups. The present invention also seeks to provide urological catheter system that is matched to different intra-abdominal pressures in different patient groups.

It is important to consider that in order to overcome the joint problems of infections and incontinence produced from current catheters, a systemic rather than a component approach needs to be adopted.

### SUMMARY OF THE INVENTION

Thus, according to a first aspect of the invention there is provided a urological device comprising one or more valves within a valve housing, external to the body when in use, with means for connection to an in-dwelling (“Foley”) catheter and a means for connection to a urine collection device (“Leg bag”) for the reduction of Catheter Associated Urinary Tract Infections (CAUTI's), the valve characterised by having a higher opening pressure than closing pressure, and the urological device having one or more valves movable from a closed position to an open position in response to applied urological/intra-abdominal pressure; and wherein the valve has a region of coaption and the type and/or number of valves is varied to provide opening pressures matched to one or more intra-abdominal pressures in one or more patient groups.

The valve may be provided with a plurality of valve leaflets possessing a region of coaption and wherein the selection of the design and number of the valves may vary the opening and closing pressure of the device. For the avoidance of doubt the term coaption or coaptation shall mean the uniting of two or more surfaces or leaflets

According to a further aspect of the invention there is provided a urological device as described herein wherein the one or more valves is selectable from a plurality of valves. The criteria for selection of the one or more valves shall include, but shall not be limited to, the weight of the patient, the gender of the patient, the mobility of the patient, etc.

Thus, in a further aspect of the invention there is provided a urological kit comprising at least a urological device as described herein, and a selection of the one or more valves as described herein.

In the urological device of the invention the one or more valves will automatically open in response to a predetermined pressure level, relative to atmospheric pressure, in the patient's bladder acting upon the valve. However, advantageously, the one or more valves will close again when the pressure level in the patient's bladder drops below the predetermined value. Thus, according to this aspect of the invention there is provided a urological device as herein described wherein the range of opening pressure is provided across an intra-abdominal pressure (P. Abd.) of from about 5 cm H₂O to <120 cm H₂O. Preferably the opening pressure is significantly higher than the closing pressure. For example, the opening pressure is at least 10% greater than the closing pressure and typically 50-100% greater. Preferred valves for use in the present invention will have an opening pressure of about 40 cm H₂O, or 50 cm H₂O or 55 cm H₂O. In a particularly preferred aspect of the invention the urological device will be provided with a selection of valves of about 40 cm H₂O, 50 cm H₂O and 55 cm H₂O, such that the patient or the medical practitioner can select the valve or valves appropriate for the patient. However, it will be understood that the person skilled in the art may select the appropriate valve pressure depending upon the patient.

In a particular aspect of the invention the urological device may be provided with a plurality of valves. Each of the plurality of valves may have the same range of opening pressure or they may be different.

Although the nature of the one or more valves may vary, a particularly preferred valve is a resiliently flexible valve, i.e. comprising a resiliently flexible diaphragm valve member. Such a valve member is described in U.S. Pat. No. 4,728,006. U.S. Pat. No. 4,728,006 describes a flexible container e.g. a shower gel container including a self-sealing dispensing valve. Generally, the valve comprises a flexible resilient diaphragm with at least one slot. This valve is closed until pressure is applied to the flexible container which causes the valve to take up a convex shape and the slit opens. The self-sealing valve described in the prior art is known to be used in, for example, containers for storage and dispensing of gels and/or viscous liquids since the resilient valve member will close when pressure is removed from the walls of the dispensing container. Such a valve is ideally suited for dispensing devices which are inverted in use.

A particularly preferred valve is a SureFlo® valve, available from LMS, Midland, Mich. 48642 USA. An alternative preferred valve is a SureSnap® valve, also available from LMS.

A particular advantage of the SureSnap® valve is that in operation the valve has a rolling action, essentially the sides of the valve invert when the valve opens. This helps the valve leaflets to stay open. Furthermore, this mimics the operation of a bladder, e.g. in a human.

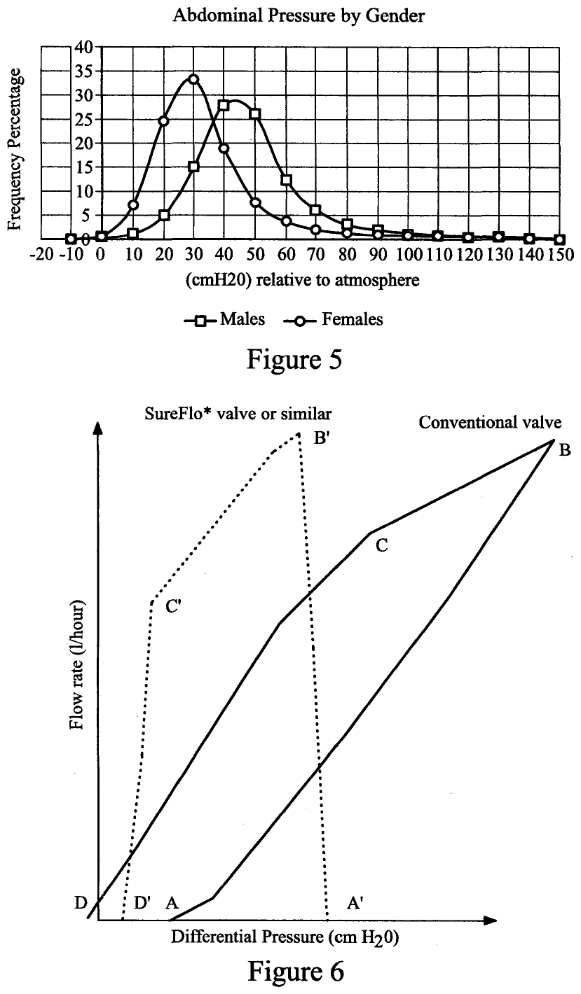

There are a wide range of valve types that might be considered for this application. Alternative valves have generic names such as “Duckbill”, “Umbrella”, “Dome” and “cross-slit”. These are available from a variety of vendors such as miniValve International of Oldenzaal in the Netherlands. FIG. 6 shows a typical pressure versus flow characteristic for a SureFlo® and a generic umbrella valve. Points A, B, C, D in FIG. 5 describe a typical umbrella valve characteristic and A′, B′, C′, D′ describe a SureFlo® valve. For a closed umbrella valve as pressure is increased there is no flow until point A. The valve will open slightly enabling a low flow (dripping). As pressure is increased, between A and B, the flow also increases with an approximate linear relationship. Once the pressure is decreased, between B to C, hysteresis is observed. The flow for decreasing pressure is higher than for increasing pressure at the same pressure. This is due to the stiffness of the rubber in the valve opening region: a high pressure is required to initially open the valve, and the valve stays open with the same effective orifice size at a lower pressure. To fully reclose certain valves a small negative pressure is required, point D. For the application in a urological device, such valves are unsuitable as they tend to drip at low pressure with the drip rate increasing with increased pressure, preventing the filling of the bladder. This contrasts with the use of a SureFlo® valve or a SureSnap® valve, which has a pressure/flow characteristic shown as A′, B′, C′, D′. As pressure is increased from zero to A′ there is no flow. At the critical opening pressure A′ the valve skirt rolls open and the pressure to keep it open is significantly reduced, due to the change in geometry afforded by the rolling action. The flow is immediately high when the valve opens. There is a critical closing pressure at C′ whereby the valve “snaps” closed: the valve skirt rolls back to the closed position. The system is reset at point D′. It will be appreciated that the SureFlo® has a pressure/flow characteristic ideally suited for use in this application with a urinary catheter: the valve does not open until a high opening pressure, flow is then instantaneously high and the valve stays open at a high flow until a significantly lower pressure when it abruptly closes, and flow stops.

A similar resiliently flexible diaphragm valve is described in U.S. Pat. No. 5,439,143. The flexible valve described in U.S. Pat. No. 5,439,143 is provided with a “rolling sleeve” such that when pressure is raised above the predetermined discharge pressure, the valve head shifts outwardly in a manner which causes the connector sleeve to double over and then extend rollingly, and thereby apply a torque to the valve head which assists in opening the orifice of the valve.

Furthermore, International Patent application No. WO 2015/040413 describes a method of simultaneously cutting and annealing an opening in a polymeric implantable medical device which provides a cut surface of low rugosity. Rugosity is generally a measure of surface roughness, therefore the lower the rugosity value, the smoother the surface. The use of a low rugosity cut surface in the valve is advantageous in that it is smooth and substantially non-adherent, e.g. non-adherent to microorganisms, such as bacterial and/or yeast cells. Infectious material is known to adhere, and build up, around rough surfaces such a catheter eyelet. These are conventionally cut mechanically.

The urological device of the invention will generally comprise a valve body which has an inlet fitment to attach to standard in-dwelling catheters. The valve body may also be provided with an outlet fitment to attach to a standard urinary receptacle (for example, a leg bag). The use of such inlet and outlet fitments is advantageous in that they allow for the rapid inclusion or replacement of the device. It also allows for the use of a wide range of existing catheters and urinary receptacles. Such a device is preferentially external to a patient's body and can be connected and disconnected to the catheter. This reduces the costs and timescales associated with regulatory approvals and minimises changes in nursing practice. If the valve were to be permanently attached to the catheter then it would be regulated as a device which enters the body and this requires a more onerous path for regulatory approval and acceptance by health care providers.

It will be appreciated by those well versed in the art that a closed system, such as that described thus far, will suffer from a syphoning action following the first opening of the valve. The negative pressure head of the urine collection device relative to the bladder will prevent closure of the valve. Hence for reliable operation the valve housing should be provided with a small vent to atmosphere. Venting is essential to allow the valve to re-close after it has been opened. Some urinary collection devices are fitted with venting valves but this cannot be guaranteed for all circumstances. The valve body must therefore include a vent. The vent can be upstream, but is preferably downstream of the valve, that is, when in use, the vent is located below the valve. It will also be appreciated that an open vent will permit the access to the lumen by bacteria which is clearly undesirable. However, in a preferred embodiment the vent is provided with a check valve. A check valve is a non-return valve that allows fluid (air when the check valve is used in a vent as herein described) to flow in one direction only. When a vent is included in a leg bag, gas permeable valves, such as GoreTex™ are known to be used. However, such gas permeable valves are not check valves and the use of GoreTex™ is found to be inferior to the use of a check valve. Examples of check valves that may suitably be used in a vent of the present invention include, but shall not be limited to, umbrella valves, ball valves, duckbill valves, and the like. A preferred check valve is an umbrella valve. Umbrella valves are known, for example, to be used in venting automotive fuel tanks. An umbrella valve will generally comprise an elastomeric valve component that includes a convex diaphragm shaped sealing disk (or umbrella shape). When mounted in a seat, the convex diaphragm flattens out against the valve seat and absorbs a certain amount of seat irregularities and creates a seal. The umbrella valve allows forward flow once the head pressure creates enough force to lift the convex diaphragm from the seat and so it will allow flow at a predetermined pressure in one way and prevent back flow immediately in the opposite way. The opening pressure of the umbrella valve can be varied. Suitable umbrella valves are available from Minivalve. When the vent is provided with an umbrella valve as described herein, the vent will be located inside a vent chamber, such that the umbrella valve, when closed, seals the vent chamber from the valve chamber. One advantage of the use of a check valve in conjunction with a vent is that, inter alia, the valve becomes tuneable to the requirements of a patient. It is preferred that a vent comprising a check valve as herein described should be included in the valve assembly. However, it is within the scope of the present invention for a vent comprising a check valve separate from the valve assembly, for example, to be included in a leg bag.

The one or more valves within a valve housing will generally comprise a urinary valve, which is a valve that allows or stops the flow of urine. Thus, such a valve should be distinguished from a vent valve described herein.

Thus, according to this aspect of the invention the urological device may be provided with one or more vents, generally, such one or more vents are positioned below the outlet, so as to prevent siphoning from the bladder to the urinary receptacle. When such a vent is provided, it may be provided with an umbrella valve.

According to a further aspect of the present invention the valve and/or the valve body may comprise an antimicrobial material, e.g. an antimicrobial polymer material. Although a variety of antimicrobial materials may suitably be used in the valve and/or the valve body, suitable antimicrobial materials may comprise a polymeric composite comprising a polymer and particles of one or more of (nano)silver, copper and keratin. Suitable polymeric materials would be known to the person skilled in the art, but may comprise polyurethane or silicon polymers. In a particular aspect of the invention the antimicrobial material comprise dispersed (nano)silver particles. The antimicrobial material may comprise a polymer material wherein the antimicrobial particles are embedded in the polymer. Alternatively, or in addition, the antimicrobial particles may be coated onto the polymer.

The urological device may also be provided with an extra lumen infection barrier comprising an antimicrobial material. The extra lumen infection barrier will generally be positioned substantially where the catheter exits a patient's body. Furthermore, the extra lumen infection barrier is shaped to match male or female anatomy.

A primary function of the urological device of this aspect of the invention is that the device provides enhanced infection and/or re-infection control, by the addition of a section antimicrobial material placed external to the patient's body. Additionally, an O-ring may also be suitable included to provide an additional barrier against the known transmission of bacteria from outside to inside the body.

Furthermore, the components of the urological device of the invention may desirably be manufactured by injection moulding. In a further preferred embodiment the urological device may be provided with a polished or mirrored finish will further impede the adherence of bacteria to the surface of the components.

The improved urological catheter system of the present invention accomplishes five main goals:
ensures all parts of the catheter system provide physical barriers and proactive anti-bacterial materials against bacterial colonisation;
ensures the system allows both voluntary and involuntary mechanisms for ‘filling and flushing’ the bladder on a regular basis;
is highly cost-effective, reduces nursing, emergency visits, etc.;
is easily adaptable to suit group or individual needs; and
has the ability to be tailored to patient groups.

Furthermore, whilst the urological catheter system can be used with a manually operated valve to empty the bladder directly into a toilet, the system also offers the advantage that the valve may be operated automatically, such that catheter system can suitably be used whether a patient is conscious or unconscious.

A particular benefit of the urological catheter system of the present invention is that patient infections can be minimised by allowing the patient's bladder to “fill and flush” which others do not, thereby reducing infections (see Example 1 herein).

According to a further aspect of the invention there is provided a urinary drainage system comprising a catheter tube comprising a balloon/anchor port and a urinary drainage channel, the urinary drainage channel being provided with one or more valves within a valve housing, with means for connection to an in-dwelling catheter and a means for connection to a urine collection device for the reduction of Catheter Associated Urinary Tract Infections (CAUTI's), the one or more valves being characterised by having a higher opening pressure than closing pressure, and the urological device having one or more valves movable from a closed position to an open position in response to applied urological/intra-abdominal pressure; and wherein the design of the coaption region and the number of valves is varied to provide opening pressures matched to one or more intra-abdominal pressures in one or more patient groups.

According to a yet further aspect of the invention there is provided a urinary catheter assembly comprising a catheter tube comprising a balloon/anchor port and a urinary drainage channel, the urinary drainage channel being provided with one or more valves within a valve housing, with means for connection to an in-dwelling catheter and a means for connection to a urine collection device for the reduction of Catheter Associated Urinary Tract Infections (CAUTI's), the one or more valves being characterised by having a higher opening pressure than closing pressure, and the urological device having one or more valves movable from a closed position to an open position in response to applied urological/intra-abdominal pressure; and wherein the design of the coaption region and the number of valves is varied to provide opening pressures matched to one or more intra-abdominal pressures in one or more patient groups.

CAUTI's may be caused by inter lumen migration of bacteria, which is prevented by the valve, or by extra lumen migration of bacteria. According to this aspect of the invention the catheter assembly may be provided with an extra lumen infection barrier, said extra lumen infection barrier being positioned external to the body of a patient substantially where the catheter exits the patient's body.

It will be understood by the person skilled in the art that extra lumen infection barrier may be positioned adjacent to the patient's body or remote from the patient's body. Furthermore, the shape of the extra lumen infection barrier may vary depending upon, inter alia, whether the catheter assembly is intended to be used on males or females.

According to a further aspect of the invention there is provided a method of urinary catheterisation of a patient, said method comprising the insertion of a urological device through the urinary tract into the bladder of a patient, said urological device comprising one or more valves and valve housing, external to the body when in use, connected to an in-dwelling catheter for the reduction of Catheter Associated Urinary Tract Infections (CAUTI's), the valve characterised by having a higher opening pressure than closing pressure, and the urological device having one or more valves movable from a closed position to an open position in response to applied urological/intra-abdominal pressure, wherein the valve is provided with a plurality of valve leaflets possessing a region of coaption; and wherein the design of the coaption region and the number of valves is varied to provide opening pressures matched to one or more intra-abdominal pressures in one or more patient groups.

An embodiment of the invention will now be described with reference to the accompanying drawings in which:

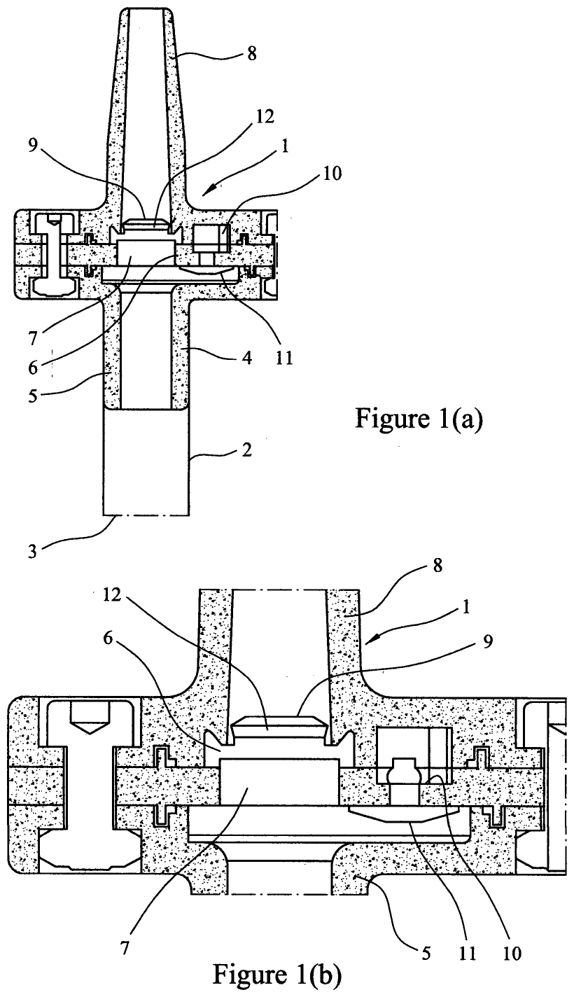

FIGS. 1(a) to (c) are cross-sectional views of a urological device valve assembly;

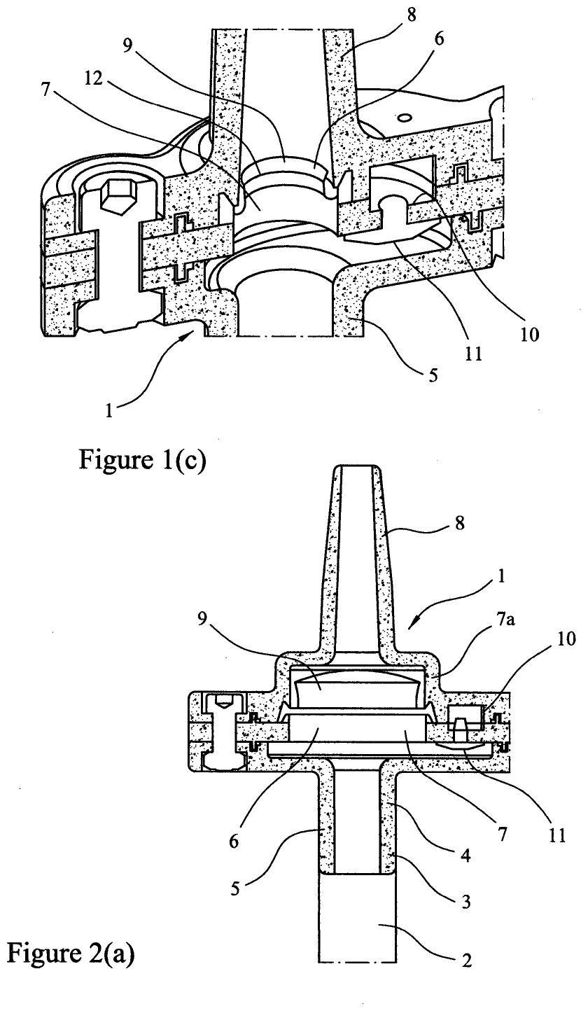

FIGS. 2(a) to (c) are cross-sectional views of a urological device valve assembly provided with a valve chamber;

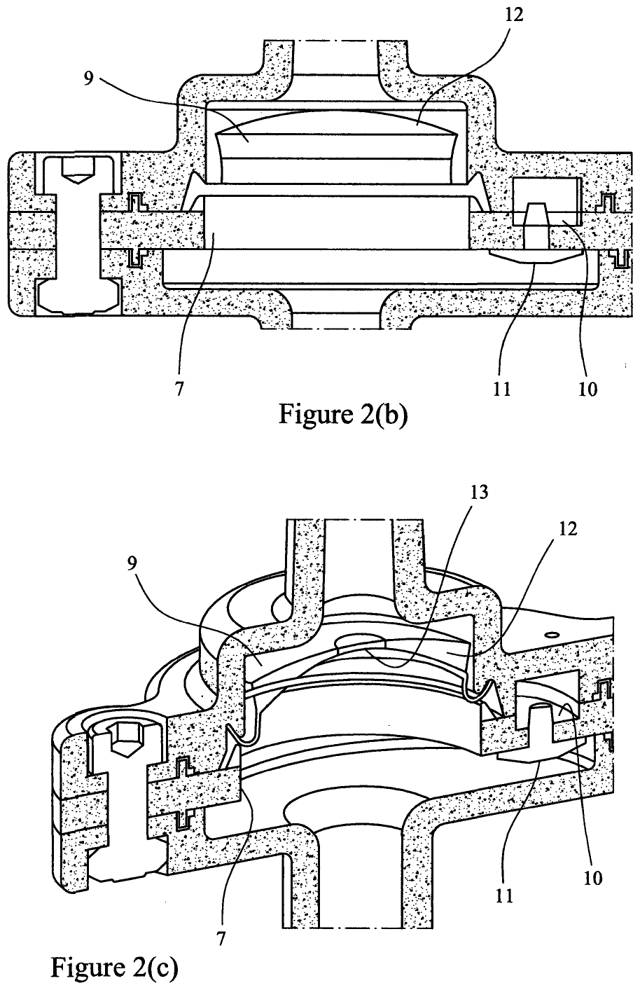

FIG. 3 a perspective view of a urological device kit;

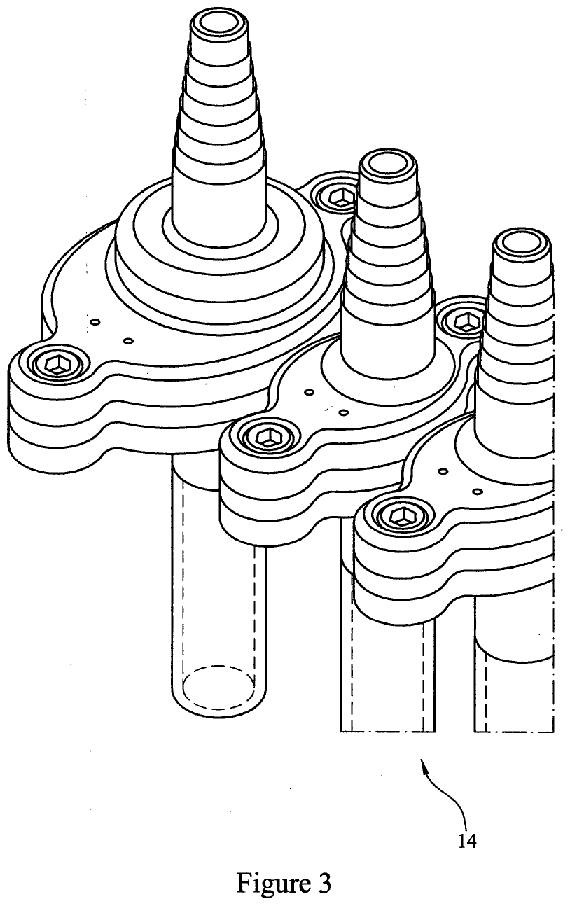

FIG. 4(a) is an enlarged, cross-sectional view of the, with the valve shown in a fully closed and partially extended position;

FIG. 4(b) is an enlarged, cross-sectional view of the valve, with the valve shown in a fully closed and fully extended position, wherein a valve head portion of which is shown beginning to snap outwardly; and

FIG. 4(c) is an enlarged, cross-sectional view of the valve shown in a fully open, and fully extended position, wherein the valve head portion of which is shown snapped fully outwardly;

FIG. 5 is a frequency plot of abdominal (bladder) pressure by gender; and

FIG. 6 shows Differential Pressure versus flow for a Sureflo™ versus an umbrella valve.

Referring to FIGS. 1(a) to (c), a urological device 1 comprises a catheter 2, wherein a first end 3 is connected to a patient (not shown) and a second end 4 is connected to a fluid inlet nozzle 5 of a valve member 6.

The valve member 6 comprises a fluid inlet nozzle 5, a valve chamber 7 and a fluid outlet tube 8. A diaphragm valve 9 is located in the valve chamber 7. The valve chamber 7 is provided with a vent 10. The vent is provided with an umbrella valve 11.

The diaphragm valve 9 comprises a concave valve member 12 consisting of a resiliently flexible material and is provided with a slit 13 (not shown, see FIG. 4) or a pair of slits in its body. It will be appreciated that a variety of slit arrangements may be suitable for the invention to perform satisfactorily.

Referring to FIGS. 2(a) to (c), a urological device 1 comprises a catheter 2, wherein a first end 3 is connected to a patient (not shown) and a second end 4 is connected to a fluid inlet nozzle 5 of a valve member 6.

The valve member 6 comprises a fluid inlet nozzle 5, a valve chamber 7 and a fluid outlet tube 8. The valve chamber 7 provides a housing 7(a) for a diaphragm valve 9. The valve chamber 7 is provided with a vent 10. The vent is provided with an umbrella valve 11.

In the urological device of both FIGS. 1(a) to (c) and FIGS. 2(a) to (c), when the concave valve member 12 is in the closed position fluid is retained behind the concave valve member 12. When the fluid pressure reaches a predetermined level the concave valve member 12 flexes and the slit 13 opens to create an aperture (not shown), allowing fluid to flow through the fluid outlet tube 8, thus relieving the fluid pressure. When the fluid pressure drops below the predetermined level, the slit 13 closes again and the valve is shut. This is shown more clearly in FIG. 4.

Referring to FIG. 3, a urological device kit 14 comprises a group of three urological device valve assemblies. Although it will be understood that in the kit of the invention the number of urological device valve assemblies may be varied, the present invention particularly provides a kit comprising urological device valve assemblies, e.g. provided with a selection of valves of about 40 cm H₂O, 50 cm H₂O and 55 cm H₂O opening pressure, which is found to be suitable for use with the majority of patients.

Referring to FIGS. 4(a) to (c), in operation, the valve functions in the following manner: When fluid pressure imparts with the valve 6, as illustrated in FIG. 4(a), valve head 14 shifts axially outwardly by rolling connector sleeve 15 over upon itself. Valve head 14 continues to extend outwardly toward the exterior of valve seat 16 until connector sleeve 15 is fully extended.

As illustrated in FIG. 4(b), valve head 14 continues to shift outwardly. However, since connector sleeve 15 is fully extended, further outward shifting of valve head 14 longitudinally tenses or stretches connector sleeve 15, thereby increasing the outward force applied to the valve head 14. Further outward movement of valve head 14 tends to flatten or straighten valve head 14, particularly along exterior surface 17, as illustrated in FIG. 4(b). This flattening motion dilates the circular configuration of valve head 14.

When connector sleeve 15 is in the fully extended position; and additional pressure is imparted on the valve 6, exterior rim 18 moves axially and radially outwardly.

The “snap” type opening of valve 6 is achieved, by the force exerted by connector sleeve 15, which is sufficient to substantially distort the shape of valve head 14. When valve 6 assumes the fully extended and fully open position illustrated in FIG. 4(c), valve flaps 19 of valve head 14 are bent or elastically deformed outwardly. Valve flaps 19 tend to fold openly and the continued radial inward compression applied to valve head 14 by connector sleeve 15 and the outwardly oriented torque applied thereto by connector sleeve 15 combine to keep valve 6 in the fully open position, even if the pressure imparted on valve 6 is reduced.

Hence, after valve 6 has been opened through the application of fluid pressure, that pressure which is required to maintain fluid flow through valve 6 is reduced, or less than the threshold pressure, so as to provide greater ease of flow control.

The resiliency of connector sleeve 15 serves to resist the dilating action of valve head 14 and therefore if the resiliency of connector sleeve 15 is varied, for example, by making connector sleeve 15 thicker or thinner, the amount or degree of snap action can be adjusted for any specific patient requirements.

When pressure is reduced, valve 6 will still remain open in substantially the fully open position shown in FIG. 4(c), until the pressure reaches the preselected closure pressure, at which point, the forces developed in connector sleeve 15 through elastic deformation from its original moulded shape, pulls valve head 14 inwardly, back into a concave orientation, thereby securely valve 6 with a snapping action.

This snap closing motion of valve head 14 serves to close valve 6 very quickly and very completely, so as to sharply cut off the stream of fluid being discharged without any drops or dribbles. Valve 6 will continue to assume the fully closed, until such time as the pressure is further reduced, due to the vent (not shown) to permit the resiliency in connector sleeve 15 to urge valve head 14 back into a fully retracted position.

Referring to FIG. 5, the graph illustrates that the urological pressure varies within patient groups and also varies depending upon gender.

Referring to FIG. 6, a typical pressure versus flow characteristic is shown for a SureFlo® and a generic umbrella valve. Points A, B, C, D in FIG. 5 describe a typical umbrella valve characteristic and A′, B′, C′, D′ describe a SureFlo® valve. For a closed umbrella valve as pressure is increased there is no flow until point A. The valve will open slightly enabling a low flow (dripping). As pressure is increased, between A and B, the flow also increases with an approximate linear relationship. Once the pressure is decreased, between B to C, hysteresis is observed.

### EXAMPLE 1

In vitro Valve Tests

Bristol Urological Institute carried out in vitro tests upon valves. The valve is able to open and close reliably for up to 14 days and prevented the migration of a highly motile bacterium, Proteus mirabilis up through the drainage system. The bladder of control models (without the valve) became infected with Pr. mirabilis between 144 and 168 hours but the bladders of the valved models remained sterile. This pilot works demonstrates that the more natural filling and emptying cycle that the valve provides reduces the risk of infection through the intraluminal route in the drainage system when compared with the normal continual drainage that happens with an indwelling catheter.

### EXAMPLE 2

Urinary Catheter ‘Fill and Flush’ (FF) Valve Trial

Human Trials—

Urinary Catheter ‘Fill and Flush’ Valve: Safety, Effectiveness, Acceptability and Feasibility Trial, Protocol Version 2.3 Dated Jul. 4, 2016 Developed and to be conducted by Prof. M. Fader, University of Southampton.

Aims—

To assess the safety, effectiveness, comfort and acceptability of the valve on acute care and community-dwelling participants with a short or long term urinary catheter.

Objective—

Determine if the process of filling and automatic draining is comfortable and acceptable for participants, if the valve opens automatically for bladder emptying (<100 ml remains) during daily activities and rest.

PPI (Patient and Public Involvement) Study Management—

Steering group of relevant patients, carers and clinicians will support this work by, for example, providing comment on study documentation and advising on refinement of recruitment strategy.

Outcomes—

Reliable valve opening, bladder emptying, comfort, acceptability, Quality of Life

Setting—

Clinical Research Facility—dedicated research area at Southampton General Hospital.

Participants—

16 of each long and short-term indwelling catheter (LTIC) users over 18 years (8 who currently use a standard manual valve (e.g. a ‘flip-flo’) and 8 who use a leg bag. Eligibility and Exclusion criteria will be applied.

Data Collection—

Demographic, Quality of Life, Number and frequency of openings, Recumbent and resting, residual bio-burden.

Valve Questionnaire—

Participants will assess the valve for comfort, discreetness, appearance, ease of use and effectiveness (including any leakage).

Randomisation—

Participants will be randomised to a Study Arm using block randomisation, indicated by a letter in a sealed envelope, with 10 participants in each Study Arm.

Microbiological Analysis—

Will be carried out on urine samples taken at the start and end of the study period, and on catheters following removal using culture onto selective agar. For catheter samples, biofilm formation will be analysed by EDIC Microscopy and indirect methods using resuspension and viability stains.

Transcripts Analysis—

Will include constant comparison aided by NVivo software, iteratively rearranged as themes develop, variations are highlighted and divergent or contradictory cases explored.

Testing Procedure
1. Bladder scan and record volume in Valve Log
2. Fit the lowest pressure valve and attach a leg bag for urine collection
3. Bladder scan and record volume in Valve Log
4. When not being scanned, the participant will be asked to sit in a chair, only standing to transfer between bed and chair.
5. Ask the participant to drink a minimum of 500 ml of fluids over first two hours, 150 ml per hour until 6 hours and freely thereafter.
6. If the valve is remaining open and urine is draining freely, replace with the next level pressure valve (up to 3 valves) until free drainage has ceased.
7. Record participant's bladder sensations before each scan using Valve Log.
8. With the participant lying down, scan bladder every hour (record time and volume in Valve Log) until volume is >400 ml or the participant experiences bladder discomfort or the valve automatically opens.
   a. If the valve automatically opens:
      i. Record the amount of urine voided
      ii. Scan the bladder for any residual volume
   b. If the volume is >400 ml or the participant is experiencing discomfort
      i. Ask the participant to contract abdominal muscles to increase bladder pressure or bear down as if trying to pass urine.
      ii. If the valve opens, proceed as above (a).
      iii. If the valve does not open, disconnect the valve, re-attached the leg bag and allow the bladder to drain.
9. If the valve has opened with a urine volume of less than 500 ml, repeat process 7 & 8 at 1 hourly intervals for 8 hours. If valve did not open at less than 500 ml, stop testing.

1. A urological device comprising one or more valves within a valve housing, external to the body when in use, with means for connection to an in-dwelling catheter and a means for connection to a urine collection device for the reduction of Catheter Associated Urinary Tract Infections (CAUTI's), the valve characterised by having a higher opening pressure than closing pressure, and the urological device having one or more valves movable from a closed position to an open position in response to applied urological/intra-abdominal pressure; and wherein the valve has a region of coaption and the type and/or number of valves is varied to provide opening pressures matched to one or more intra-abdominal pressures in one or more patient groups; and wherein said valve housing is provided with a vent.

2. (canceled)

3. A urological device according to claim 1 wherein the vent is provided with a check valve.

4-9. (canceled)

10. A urological device according to claim 1 wherein the valve comprises a resiliently flexible diaphragm valve member.

11. A urological device according to claim 10 wherein the diaphragm valve comprises a resiliently flexible material provided with a slit in its body.

12. (canceled)

13. A urological device according to claim 1 wherein the valve is provided with a “rolling sleeve”.

14. A urological device according to claim 11 wherein the resiliently flexible diaphragm valve member is cut to provide a plurality of valve leaflets; and wherein the valve leaflets are cut to provide a low rugosity cut surface.

15. A urological device according to claim 14 wherein the valve leaflets are cut by ultrasonic cutting.

16. A urological device according to claim 1 wherein the valve or valve body comprises an antimicrobial material.

17. A urological device according to claim 16 wherein the antimicrobial material comprises a composite of a polymeric material and particles of one or more of (nano)silver, copper and keratin.

18.-28. (canceled)

29. A urinary drainage system comprising a catheter tube comprising a balloon/anchor port and a urinary drainage channel, the urinary drainage channel being provided with one or more valves within a valve housing, with means for connection to an in-dwelling catheter and a means for connection to a urine collection device for the reduction of Catheter Associated Urinary Tract Infections (CAUTI's), the one or more valves being characterised by having a higher opening pressure than closing pressure, and the urological device having one or more valves movable from a closed position to an open position in response to applied urological/intra-abdominal pressure; and wherein the valve has a region of coaption and the type and/or number of valves is varied to provide opening pressures matched to one or more intra-abdominal pressures in one or more patient groups; and wherein said valve housing is provided with a vent.

30. (canceled)

31. A urinary drainage system according to claim 29 wherein the vent is provided with a check valve.

32.-35. (canceled)

36. A urinary drainage system according to claim 29 wherein the valve or valve body comprises an antimicrobial material.

37. A method of urinary catheterisation of a patient, said method comprising the insertion of a urological device through the urinary tract into the bladder of a patient, said urological device comprising one or more valves and valve housing, external to the body when in use, connected to an in-dwelling catheter for the reduction of Catheter Associated Urinary Tract Infections (CAUTI's), the valve characterised by having a higher opening pressure than closing pressure, and the urological device having one or more valves movable from a closed position to an open position in response to applied urological/intra-abdominal pressure, wherein the valve is provided with a plurality of valve leaflets possessing a region of coaption; and wherein the design of the coaption region and the type and/or number of valves is varied to provide opening pressures matched to one or more intra-abdominal pressures in one or more patient groups; and wherein said valve housing is provided with a vent.

38. (canceled)

39. A method according to claim 37 wherein the vent is provided with a check valve.

40.-42. (canceled)

43. A method according to claim 37 wherein the one or more valves comprise a resiliently flexible diaphragm valve member.

44. A urological device according to claim 43 wherein the diaphragm valve comprises a resiliently flexible material provided with a slit in its body.

45-46. (canceled)

47. A method according to claim 44 wherein the resiliently flexible diaphragm valve member is cut to provide a plurality of valve leaflets; and wherein the valve leaflets are cut to provide a low rugosity cut surface.

48. A method according to claim 47 wherein valve leaflets are cut by ultrasonic cutting.

49. A method according to claim 37 wherein the valve or valve body comprises an antimicrobial material.

50. A method according to claim 49 wherein the antimicrobial material comprises a composite of a polymeric material and particles of one or more of (nano)silver, copper and keratin.

51.-57. (canceled)
